(19) United States
(12) Patent Application Publication
(10) Pub. No.: US 2015/0064308 A1
(43) Pub. Date: Mar. 5, 2015

(54) Protein Recovery

(71) Applicant: Peter Edmond Vaughan Williams, Northamptonshire (GB)

(72) Inventor: Peter Edmond Vaughan Williams, Northamptonshire (GB)

(73) Assignee: AB AGRI LIMITED, London (GB)

(21) Appl. No.: 14/237,984

(22) PCT Filed: Aug. 10, 2012
(86) PCT No.: PCT/GB2012/000655

§ 371 Date: Jun. 23, 2014

(30) Foreign Application Priority Data
Aug. 10, 2011 (GB) 1113811.2

(51) Int. Cl. A23K 1/06; C12P 7/06

(52) U.S. Cl. CPC A23K 1/06; C12P 7/06
USPC 426/54; 435/161; 435/289.1; 530/350; 426/624

(57) ABSTRACT

A process for recovering a proteinaceous material from a fermentable organic material, comprising: (i) forming an aqueous mixture of a fermentable organic material and a fermentation agent capable of fermenting the organic material to produce ethanol, wherein the organic material is a vegetable material containing protein; (ii) fermenting the aqueous mixture to produce ethanol; (iii) recovering from the fermented aqueous mixture an ethanol stream which is rich in ethanol and a co-product stream comprising unfermented organic material, fermentation agent and an aqueous solution of dissolved solids in water; (iv) subjecting the co-product stream to a first separation stage to recover a first stream rich in the unfermented organic material and a second stream rich in the fermentation agent suspended in the aqueous solution; and (v) subjecting the first stream to a protein recovery step in which at least part of the fibre in the unfermented organic material is separated from the unfermented organic material to provide a protein rich material and a residual fibre containing less protein than the rich material.

The present invention relates to recovery of co-products from an ethanol fermentation process, such as a bio ethanol process. The co-products are particularly useful in animal feed material.

It has been known for many years to use bio ethanol refineries to convert biological material into useful chemical products. In a typical bio refinery a plant material, such as grain containing starch (e.g. wheat or maize) is treated to produce ethanol (so-called “bio ethanol”). The process can be used to produce both potable alcohol and industrial ethanol

For example, in a conventional bio ethanol plant, wheat is fermented using yeast as the fermentation organism to produce bio ethanol as a main product and co-products, such as animal feed. The ratio of bio ethanol to co-products is approximately 1:1, on a weight basis.

The principle co-product of a bio ethanol plant is called dried distillers grains and solubles (“DDGS”). DDGS is used in the animal feed market, primarily as a feedstock for ruminants. In a conventional process, after the ethanol has been produced by fermentation, it is separated from the fermentation products by distillation.

The residue after distillation of the ethanol (termed whole stillage) is then dried to produce the co-product, DDGS. To aid in dying, the whole stillage is separated into two fractions, a solids fraction and a liquid fraction. This first separation may be carried out in a decanter to produce a solid and a liquid output. The solid output may be pressed into a cake. The liquid output is subjected to evaporation to make a syrup containing, among other things, yeast—this syrup is known as condensed distiller's solubles (CDS). The CDS is then added to the pressed cake and dried to form what is known as the DDGS.

Although the bio ethanol process has been used for many years, very little work has been done on further development of the co-products of the process, and there is very little published information about co-product development.

Current wheat bioethanol refineries use a limited range of plant designs, in which the co-product streams are combined to form a single product, DDGS. Much of the development work on DDGS has focused on the dried product when the protein has been heat treated and is often inexorably bound in Maillard type products.

We refer to Bioresource Technology 100 (2009) 5876-5883 (article entitled “Integrating alkaline extraction of proteins with enzymatic hydrolysis of cellulose from wet distiller's grains and solubles” by Bals et al); and Bioresource Technology 101 (2010) 5444-5448 (article entitled “An attempt towards simultaneous biobased solvent based extraction of proteins and enzymatic saccharification of cellulosic materials from distillers grains and solubles” by Dastta et al). These documents are directed to the treatment of DDGS, which is the product of a conventional bio ethanol processes.

In a paper by J. Knott and K Shurson (Effects of feeding diets containing spray dried corn condensed distillers solubles (CDS) and associated fractions on growth performance of early-weaned pigs. J. Knott, G. Shurson, M. Hathaway and L. Johnston J. Anim Sci. Vol 83 (Suppl. 2) p. 71 Ethanol Byproduct may be a diet alternative. National Hog Farmer. Feb. 15, 2005) work was done on the CDS co-product of bio ethanol plants. The authors separated the CDS into two products, yeast cream (YC) and residual solubles (RS). The products were tested for utility as feed additives in animal feed. The purpose of the study was specifically to test whether the co-products of the bio ethanol process had any utility as growth factors.

The Knott/Shurson paper describes subjecting the CDS to a spray drying process to separate the CDS into three fractions, called “sprayed dried distiller's solubles”, “spray dried yeast cream”, and “spray dried residual solubles”. This process would not be suitable for the large scale recovery of yeast, because its cost would be prohibitive. Furthermore, based on the disclosure of the Knott/Shurson paper, there would be no motivation for the skilled person to seek to recover yeast on a large scale, as the paper is concerned with the use of small quantities of yeast as a growth factor present in the mixture, and does not make any reference to the amount of yeast which may be available for recovery from the CDS or the large scale recovery of yeast per se.

It is known that yeast is a co-product of the bio ethanol process, and indeed this is inevitable, as the process itself relies on the presence of yeast for the fermentation.

Yeast has been used in the animal feed market for many years, when as a dead yeast as feed material and live yeast as a feed additive. Yeast has a high content of digestible protein, and is therefore potentially useful as a feed material for use in animal feed. However, to date, yeast has not been used to any significant extent as a feed material, owing to the lack of availability of sufficient quantities of the material at a cost effective price compared to other feed materials. In commercially available animal feeds, there is a wide range of high protein feed materials of which, rape meal, soya bean meal and fishmeal are primary examples. There are some examples of dried yeast being used on a commercial basis as a feed material for fish, but it has not been used for animals, such as ruminants (cattle and sheep) or mono-gastrics, such as pigs. Yeast is mostly used as a feed additive in amounts generally less than 2 wt % of the total weight of animal feed when it is used with relevant claims to improve the performance of animals.

It would be desirable to use yeast as a feed material for supplying digestible protein to animals, but except in certain limited circumstances (such as the aquatic use mentioned above), it is not feasible to do so. This is because of the cost of the available yeast is too high.

During the bio ethanol fermentation process, the yeast is added into the mixture after saccharification in order to ferment the carbohydrate substrate. The quantity of yeast added may be large and sufficient to ferment the substrate available or may be added in a smaller quantity and allowed to multiply in the medium to the point at which there is sufficient yeast to ferment the total available carbohydrate.

A process for the recovery of yeast has been described in our International Patent Publication Number WO2010/109203. In that publication, we described that the quantity of yeast produced as a co-product of the bio ethanol production process is much higher than had been appreciated. We also described that the amount of yeast produced as a co-product is in the range 10-20 wt %, based on the total weight of the co-products. This typically represents about 4-7 wt % of the total output mass from the process, which is much higher than the amount which would be expected.

We also described that a new yeast containing composition can be recovered from a bio ethanol process, which has an unexpected positive effect on the growth of animals.

Ethanol had been produced from bio ethanol plants on a large scale for the past 15 to 20 years. For example, the European annual capacity for bioethanol production in 2008 had risen to over 4 billion litres. However, in the prior art, the yeast fraction had never been recovered on a commercial scale either from the process itself or from co-products of the bio ethanol process, because it had not been appreciated that there was a significant amount of it present, or that it could be efficiently recovered. Prior to the processes described in WO2010/109203, the yeast was not available at a price or quantity such that it could be conveniently used as a feed material in animal feed.

The present invention relates to the treatment of the residual fibre fraction produced in an ethanol fermentation process.

According to one aspect of the invention there is provided a process for recovering a protein rich material from a fermentable organic material, comprising:
(i) forming an aqueous mixture of a fermentable organic material and a fermentation agent capable of fermenting the organic material to produce ethanol, wherein the organic material is a vegetable material containing protein;
(ii) fermenting the aqueous mixture to produce ethanol;
(iii) recovering from the fermented aqueous mixture an ethanol stream which is rich in ethanol and a co-product stream comprising unfermented organic material, fermentation agent and an aqueous solution of dissolved solids in water;
(iv) subjecting the co-product stream to a first separation stage to recover a first stream rich in the unfermented organic material and a second stream rich in the fermentation agent suspended in the aqueous solution; and
(v) subjecting the first stream to a protein recovery step in which at least part of the fibre in the unfermented organic material is separated from the unfermented organic material to provide a protein rich material and a residual fibre containing less protein than the rich material. Advantageously, step (v) comprises subjecting the first stream to a protein concentration step in which at least part of the fibre in the unfermented organic material is dissolved and separated from the unfermented organic material to leave a protein rich material and a residual soluble fibre containing less protein than the proteinaceous material.

The production and recovery of the protein-rich unfermented organic material may be achieved by any means for removing the fibre from the unfermented organic material. Suitable means include physical treatments, including heat treatment; and chemical treatments, such as hydrolysis treatment with for example, caustic soda or any other means of dissolving the fibre fraction.

It is preferred that the fibre is separated from unfermented organic material by solubilising non-protein containing fibre in the unfermented organic material, then removing the solubilised fibre from the residual proteinaceous material, preferably using any suitable means for separating a liquid from a solid.

It is particularly preferred that the residual fibre is solubilised by hydrolysis, most preferably by enzymatic hydrolysis. Suitable enzymes for enzymatic hydrolysis are a wide range of cellulosic enzymes.

In a preferred embodiment, the process according to the invention also includes the recovery of the fermentation agent. Therefore, the process preferably includes the step of subjecting the second stream to a second separation step, capable of recovering suspended solids from a liquid, to recover a third stream rich in the fermentation agent and a fourth stream rich in the aqueous solution; and, if necessary, drying the third stream to recover a composition comprising the fermentation agent.

The first separation stage is preferably a decanting step, i.e., the separation is carried out in a decanter.

In prior art processes, the first stream would subsequently be combined with the second stream without any separation steps (such as defined in step (v)) other than drying having been carried out on the first or second streams.

It is an advantageous feature of the present invention that the first stream is subjected to the treatment of step (v) without any intervening treatment steps.

It is an advantageous feature of the invention that substantially no part of the second stream is combined with the first stream prior to step (v). However, in certain embodiments, it may be possible for the fourth stream to be combined with the first stream prior to step (v).

It is a particularly advantageous feature of the invention that step (v) is carried out after step (iv) without subjecting the first stream to a drying step and/or a heating step.

In an embodiment, the solubilised residual fibre may be combined with the fourth stream which is rich in the aqueous solution. This may be done before or after step (v), but it is preferably after step (v).

The residual solubilised fibre, optionally in combination with the contents of the fourth stream, may be recycled to the fermentation step (ii).

The fermentation agent may be any agent used in the fermentation of organic materials to produce ethanol. In one embodiment, the fermentation agent comprises, and more preferably consists of, fungal cells. More specifically, the fungal cells comprise, and more preferably consist of single-celled ascomycetous fungal cells, particularly yeast. In the preferred embodiment, the yeast is of the genus Saccharomyces. Yeast of the genus Saccharomyces Carlsbergiensis is particularly suitable.

In the following description, the process will be described with particular reference to processes using yeast as the fermentation agent, but it will be appreciated that this description is equally applicable to the use of protein containing fermentation agents, including microbial protein-containing cells other than yeast. Furthermore, in another embodiment, the fermentation agent may be a bacterial fermentation agent, such as zymomonas mobilis. The process according to the invention is suitable for any process for the fermentation of organic material to form ethanol (which may be ethanol for industrial use, or potable ethanol). In general, by “fermentation” is meant the biological process by which sugars, such as glucose, fructose and sucrose are converted to carbon dioxide and ethanol.

In accordance with conventional processes, the ethanol stream may be separated from the co-product stream by distillation.

Prior to the publication of WO2010/109203, it had not previously been appreciated that the yeast is present in the co-product stream as a suspension, and that it may be separated from the co-product stream by any process suitable for removing a solid suspension from a liquid. In an advantageous embodiment, the separation process is a mechanical separation process, in particular centrifugation. One particularly advantageous process for separating the yeast from the other co-products is known as disk stack separation which employs centrifugal force to separate particulate matter from a liquid. The technique of disk stack separation, per se, is known in the art, but it has not previously been applied to the process according to the invention.

The co-product stream is known in the art as “whole stillage”. It comprises predominantly water, undissolved unfermented protein-containing organic material and undissolved fermentation agent, such as yeast. It also contains non-starch polysaccharides. For example, when the fermented organic material is wheat, the whole stillage contains NSPs based on arabinose, urinic acid, glucan, xylose and glucose residues and also contains glucomannan. The NSPs in wheat are approximately 25 wt % water soluble and 75 wt % water insoluble. Of the soluble fraction over 90 wt % of the NSPs are arabinoxylan or beta-glucan, with the remainder being galactose. The water is an aqueous solution containing dissolved solids, including unfermented soluble organic material. According to an embodiment of the invention, the undissolved unfermented organic material, which is typically of a fibrous consistency, is separated from the rest of the whole stillage in a first separation step, leaving the aqueous solution and the fermentation agent. It will be appreciated that the unfermented organic material separated from the rest of the whole stillage will still contain some fermentation agent and some of the aqueous solution. However, the majority of the fermentation agent and the aqueous solution is separated from the undissolved unfermented material in the first stage of the separation. The undissolved unfermented organic material may contain a useful amount of the fermentation agent, such as yeast. Therefore, if desired, part of the recovered undissolved unfermented organic material may be recycled back into the co-product stream to improve the yield of fermentation agent.

As mentioned above, prior to the publication of WO2010/109203, it had not previously been recognised that the fermentation agent, in particular the yeast, is suspended in the aqueous solution and can be readily separation by a mechanical separation technique, or equivalent. Thus, the majority of the fermentation agent, in particular the yeast, may be separated from the aqueous solution. However the recovered fermentation agent, in particular the yeast, does usually include some of the aqueous solution (including dissolved solids such as soluble non-starch polysaccharides), and therefore it is preferably dried after recovery.

In a preferred embodiment, the third stream is subjected to a dewatering step. The dewatering step preferably comprises a mechanical dewatering step. The mechanical dewatering step preferably comprises subjecting the third stream to a filter press. It is preferred that the third stream is further dried, preferably by evaporation, preferably with heating, after the dewatering step.

It is particularly advantageous that the fermentation agent, in particular the yeast, is separated from the stillage prior to subjecting the stillage or fermentation agent to any drying or evaporation step. However, it is possible, to dry the stillage, including the yeast, prior to any separation step, then to wet it again, by adding water, when it is desired to separate the fermentation agent from the stillage. This may be useful, for example, when it is desired to separate the fermentation agent from the stillage at a different location from the bin refinery.

According to another aspect of the invention there is provided apparatus for producing ethanol and a protein-containing fermentation agent comprising:
(i) a fermentation stage for fermenting an aqueous mixture comprising an organic material and a fermentation agent capable of fermenting the organic material, to produce ethanol;
(ii) a first separation stage for recovering the ethanol from the unfermented aqueous mixture;
(iii) a second separation stage, downstream of the first separation stage for recovering unfermented organic material from the fermentation agent and from an aqueous solution of dissolved solids in water; and
(iv) an organic material separation stage for separating at least some protein in the unfermented organic material from the second stage from the rest of the unfermented organic material from the second stage.

Advantageously step (iv) comprises an organic material separation stage for solubilising fibre to produce a protein rich residue of unfermented organic material from the second stage from the rest of the unfermented organic material from the second stage.

Preferably the apparatus further comprises:
(v) a third separation stage, downstream of the second separation stage, for recovering the fermentation agent from the aqueous solution. Optionally, a drier is provided for drying the third stream.

The starting material for the process may be any organic material (in particular, a starch-containing vegetable material or a cellulose-containing material, both materials also containing protein) capable of being fermented with the fermentation agent to produce ethanol. Thus, the organic material may be any fermentable vegetable, in particular any fermentable ground vegetable. Thus, the starting material may be a cereal grain, such as maize, wheat, sorghum or barley, or may be potato, cassava or beet. Alternatively, the organic material may be straw, wood or corn stover. The ethanol output may be of a grade used for industrial or fuel use, or it may be of a grade used for human consumption, such as a variety of whisky. It is especially preferred that the fermentable organic material is wheat.

It will be appreciated that the fermentation agent may, and usually will, alter in nature during the course of the process. In general, the fermentation agent used in the fermentation step is “unspent”, whereby it is capable of fermenting the organic material. The fermentation agent in the co-product stream may be a mixture of spent and unspent fermentation agent, and is usually substantially entirely spent fermentation agent.

Thus, when the fermentation agent is yeast, unspent (or “live”) yeast will be employed during the fermentation process, and by the end of the process, when recovered in the co-product stream, some or all of the yeast will be spent (or “dead”) yeast.

In this specification, the expression “fermentation agent” may refer to unspent or spent fermentation agent, and the expression “yeast” may refer to unspent or spent yeast. The composition will be clear to a person skilled in the art from the context in which the terms are used.

The recovered fermentation agent, especially the yeast (typical examples Saccharomyces cerevisiae; Saccharomyces Carlsbergiensis) produced by the process or apparatus according to the invention may be formulated for any desired end use, and may be formulated for use as a micronutrient feed additive. However, it is particularly preferred that the fermentation agent, especially the yeast, produced by the process according to the invention is formulated as a feed material in an animal feed composition. The fermentation agent, especially the yeast, may be as a feed material for ruminant animals, such as cattle, sheep and goats. It is particularly preferred that the feed material containing the fermentation agent, especially the yeast, is formulated to feed monogastric animals, such as pigs, poultry, fish, crustacea and companion animals, such as horses, cats and dogs.

The main components of the organic dry matter of food are defined as carbohydrates, lipids, proteins, nucleic acids, organic acids and vitamins (Animal Nutrition, third edition, P. McDonald, R. A. Edwards and J. F. D. Greenhalgh ISBN 0-582-44399-7). Typically, the fermentation agent, especially the yeast, would be formulated in an animal feed composition in the range from 2 to 40 wt %, preferably 3 to 40 wt %, more preferably 4 to 40 wt %, still more preferably 5 to 40 wt %, with the remainder comprising those components as defined above. In addition, the food may contain a wide range of additives which according to the definition are feed materials which have some special effect e.g. provide enhanced performance. The protein in the feed material may be provided partly or entirely by the fermentation agent, especially the yeast, produced by the process or apparatus according to the invention.

Other ingredients, such as selected amino acids (such as lysine, methionine and so on), and vitamins (such as A, D, E and so on), minerals (such as calcium, phosphorus and so on) and antibiotics may also be present in the composition.

The process and apparatus according to the present invention produce a high value protein-containing composition as a co-product, rather than the relatively low value DDGS co-product produced in the prior art. The protein-containing composition can be produced on a scale large enough to enable it to be used as a feed material in animal feed.

The invention may be used to form protein compositions based either on the proteinaceous material recovered from the process or based on the yeast recovered from the process, or based on a mixture of the two. The protein compositions formed in accordance with the invention may be included in dietary formulations for livestock as an alternative source of protein to replace a range of protein materials that are currently used either individually or in a mixture in feed (e.g. fishmeal; soya bean meal; rapeseed meal; maize gluten meal; pea protein). As such the protein composition could replace from 0.5% to 100% of the individual proteins or mixture of proteins in the diet. Preferably the protein composition according to the invention may replace about 5 to 40 wt % of the proteins in the proteins in the diet, more preferably about 20 to 35 wt %. Typically, the protein composition according to the invention may replace about 30 wt % of the proteins in the diet—this is especially appropriate for fish.

Over a wide range of species (pigs, poultry, fish) the intake per unit metabolic weight (W0.75) of the protein composition according to the invention may range from 0.01 to 90 g dry matter/W0.75/day.

The process according the invention makes it possible to produce a high value proteinaceous compositions in place of the DDGS conventionally produced in ethanol fermentation processes. The conventional DDGS, owing to its high fibre content, is generally only suitable as a feed for ruminant animals, and not for monogastric animals, such as pigs, poultry, fish, crustacea and companion animals, such as horses, cats and dogs. The proteinaceous material according to the invention has lower fibre content than DDGS, and the protein is more freely available, making it suitable as a feed for monogastric animals.

The protein content of DDGS is typically around 33 wt %. The process according to the invention, involving solubilising fibre from residual fibre to produce a proteinaceous material results in the proteinaceous material having a protein content of 40 wt % or more, for example, 50 wt % or more, or 70 wt % or more. It will of course be appreciated that the proteinaceous material does not necessarily have a protein content of 100 wt %.

The present invention makes it possible to produce two new high protein concentration co-products from a bioethanol refinery (yeast and wheat protein concentrates), which are alternatives to the DDGS conventionally produced in biorefinery processes. The new co-products can substitute for soya bean meal in livestock diets more valuable economically and produced at lower energy cost, compared with DUOS. This is achieved by separating the high quality protein from the fibrous fraction of the co-product stream.

By removing much of the fibre from the DDG (preferably using a combination of enzymatic, chemical and physico-thermal processing approaches), the protein concentration of the co-product can be raised to greater than 40%. The process increases the options for use of the product in feed formulation expanding the market options from the current use mainly in ruminants to encompass all sectors of feed production including monogastrics and high value aquaculture.

Thus, the present invention takes an innovative approach of producing two co-products by focusing on the upstream liquid and semi liquid fractions. These fractions are produced using existing equipment. At this point in the process the co-product has typically passed through mashing, fermentation and distillation process steps and is in a liquid/semi liquid phase. Yeast as a suspended solid may be separated from the solubles fraction by centrifugation. The remaining solids fraction (30% dry material) may be treated separately, to reduce its fibre content.

The second innovative step is the application of separation techniques, especially hydrolytic separation techniques (enzymes, pressure, heat, acidification) to solubilise the fibrous fraction and increase the concentration of protein in the residue.

Due to the previous saccharification, fermentation and distillation processes the wheat cell wall polysaccharides (principally cellulose and arabinoxylans) can be hydrated and open in structure, and which makes them even more amenable to enzymatic digestion and/or chemical modification. Having removed the spent yeast and lowered the fibre content, the drying costs for the residual WPC will be reduced, and the enzymatically solubilised sugars released from hydrolysis of the fibre can be transferred directly to an anaerobic digestion process, or back into ethanol fermentation.

Reference is now made to the accompanying drawings, in which:

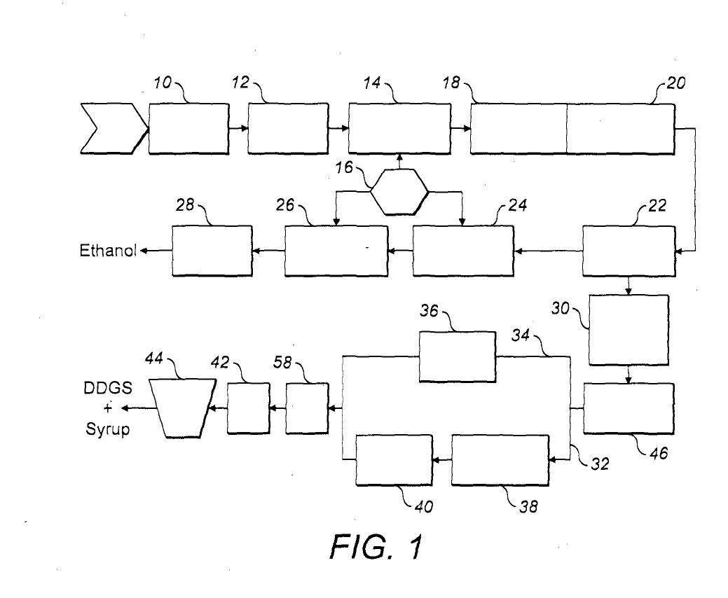

FIG. 1 is a schematic drawing of an embodiment of a bio ethanol process according to the prior art;

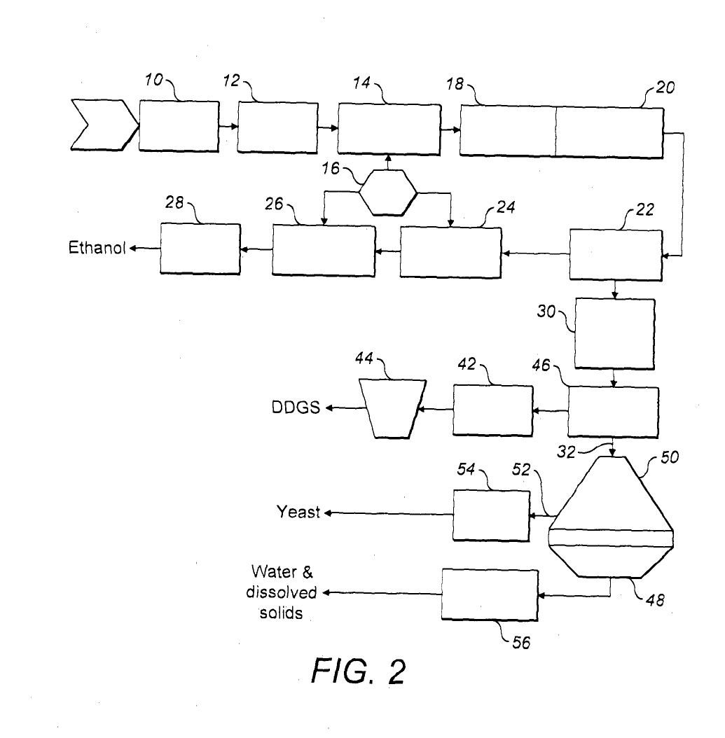

FIG. 2 is a schematic drawing of an embodiment of a process for recovering fermentation agent, in particular yeast, according to WO2010/109203;

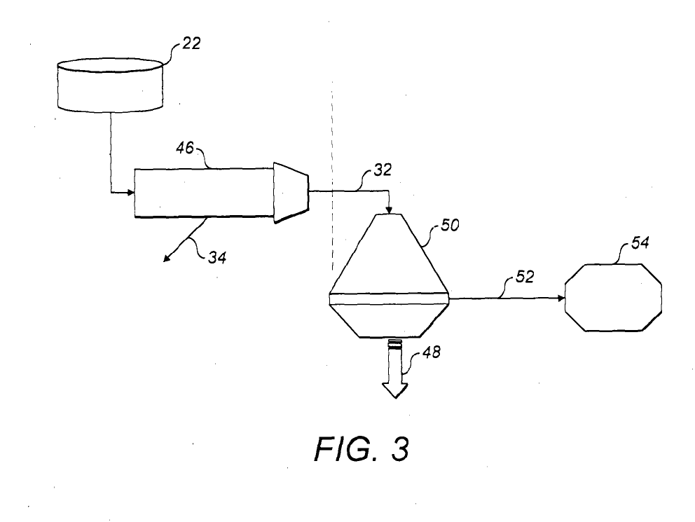

FIG. 3 is a more detailed drawing of part of the process shown in FIG. 2;

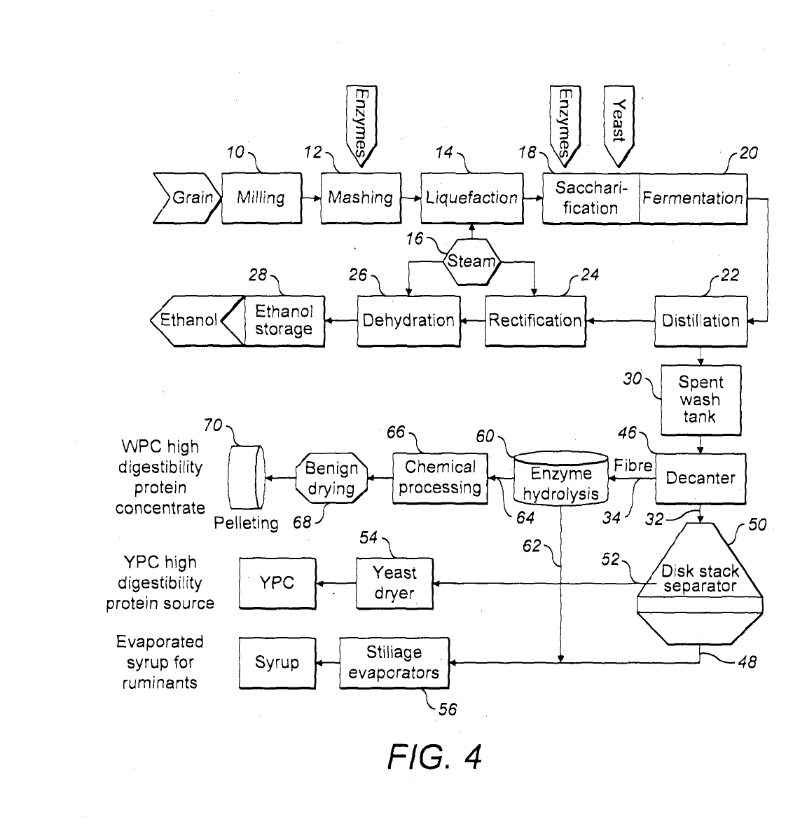

FIG. 4 is a schematic drawing of an embodiment of a process for recovering proteinaceous material according to the invention; and

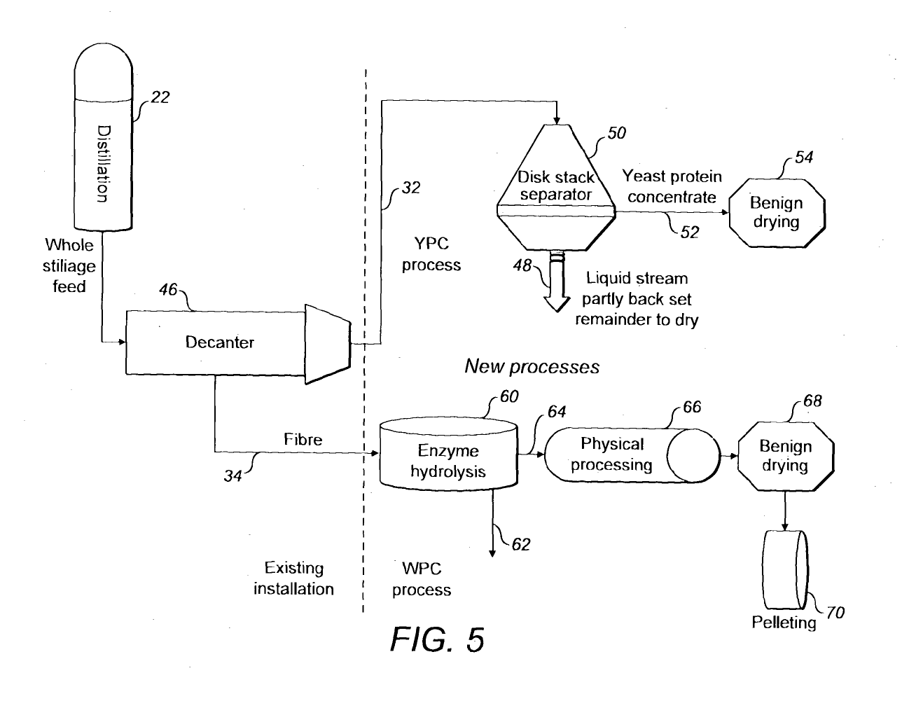

FIG. 5 is a more detailed drawing of part of the process shown in FIG. 4.

In the following description FIG. 1 represents the state of the art prior to the disclosure of WO2010/109203, while FIGS. 2 and 3 show the process described in WO2010/109203. This prior art will be described before the invention is described in order to help put the invention into context.

With reference to FIG. 1, a source of fermentable carbohydrate, more particularly a source of vegetable starch, such as wheat or maize, is fed to a milling stage 10, then slurried with water to form a mash in a mashing stage 12. The first step in starch breakdown involves saccharification, typically using α-amylase and steam. This is followed by a liquefaction stage 14, using steam from a stage 16. Further enzymes (e.g., gluco amylase) are added in a saccharification stage 18, and yeast is added in fermentation stage 20.

The fermentation produces ethanol and co-products which are discharged to a distillation stage 22, in which the majority of the ethanol is separated by distillation from the majority of the co-products. One output from the distillation stage 22 is an ethanol rich stream, which is fed to a rectification stage 24, in which the ethanol is further purified. Steam from stage 16 is also fed to the rectification stage 24.

The purified ethanol from the rectification stage 24 is fed to a dehydration stage 26, to which further steam from the stage 16 is added. The output from the dehydration stage 26 is discharged to an ethanol storage stage 28.

The co-products from the distillation stage 22, known as whole stillage, are fed to a spent wash tank stage 30, and subsequently to a decanter 32, which separates the solid unfermented organic material from an aqueous phase comprising mostly water and yeast.

The solids output 34 from the decanter 46 is pressed into a cake in a compression stage 36. The liquid output 32 from the decanter 46 is fed to an evaporation stage 38, which removes some water, followed by a further heating stage 40, which removes more water to produce a syrup. This syrup typically has a moisture content of 75 wt % water. The syrup from stage 40 is sprayed onto the cake in a stage 58, and the resultant sprayed cake is fed to a drying stage 42. The output from the drying stage 42 is DDGS, which is fed to a pelleting stage 44, which may also include a cooling stage.

The process shown in the drawings is known as a “dry-grind” process, and this is the preferred process. An alternative process, known as a “wet-grind” process may be used instead, in which an amount of fibre is separated from the starch source prior to fermentation.

Referring now to FIGS. 2 and 3, the process according to WO2010/109203 is shown. Many of the stages used in the process according to the invention may be identical to the stages shown in FIG. 1, and like parts have been designated with like reference numerals.

The solids output from the decanter 46, is still pressed into a cake [MSOffice] then dried and pelletised in stage 42 and 44.

We have found that the liquid output from the decanter comprises a large quantity of yeast suspended in water, and that the yeast can be recovered from the water in a simple mechanical separator. Recovery of yeast at this stage has not been previously contemplated. Thus, the liquid output is fed to a disk stack separator 50 which separates the yeast from the liquid. The yeast is produced in a stream 52, which is fed to a yeast drier 54. The water is produced in a stream 48, which is fed to evaporators 56 to produce a syrup. This syrup may be sprayed onto the DDGS cake, as described with respect to FIG. 1.

Referring now to FIGS. 4 and 5, the process according to the invention is shown. Many of the stages used in the process according to the invention may be identical to the stages shown in FIG. 1, and like parts have been designated with like reference numerals.

The invention in FIGS. 4 and 5 include an additional component for treating the solids output 34 from the decanter 46. The solids output 34 comprises unfermented organic material, in particular a protein-containing fibrous material. The solids are passed to an enzyme hydrolysis stage 60, in which the fibre is solubilised to produce residual proteinaceous material. The solubilised residual fibre is separated through a stream 62 and added to the stream 48. The non-solubilised material in the stage 60 is a proteinaceous material, which has higher protein content than the fibre in the stream 34. The proteinaceous material exits the stage 60 in stream 64 and is subjected to optional chemical and or physical processing in a stage 66. The proteinaceous material is then dried in a drying stage 68, and may be pelleted in a stage 70.

It will be appreciated that the invention described above may be modified in accordance with the following claims.

1. A process for recovering a protein from a fermentable organic material, comprising:
(i) forming an aqueous mixture of a fermentable organic material and a fermentation agent capable of fermenting the organic material to produce ethanol, wherein the organic material is a vegetable material containing protein;
(ii) fermenting the aqueous mixture to produce ethanol;
(iii) recovering from the fermented aqueous mixture an ethanol stream which is rich in ethanol and a co-product stream comprising unfermented organic material, fermentation agent and an aqueous solution of dissolved solids in water;
(iv) subjecting the co-product stream to a first separation stage to recover a first stream rich in the unfermented organic material and a second stream rich in the fermentation agent suspended in the aqueous solution; and
(v) subjecting the first stream to a protein recovery step in which at least part of the fibre in the unfermented organic material is separated from the unfermented organic material to provide a protein rich material and a residual fibre containing less protein than the rich material.

2. A method according to claim 1, wherein said soluble fibre is separated from the proteinaceous material by solubilising the residual fibre in the unfermented organic material, then removing the solubilised residual fibre from the proteinaceous material.

3. A method according to claim 1 or 2, wherein said residual fibre is solubilised by hydrolysis.

4. A method according to claim 1, 2 or 3, wherein said residual fibre is solubilised by enzymatic hydrolysis.

5. A process according to any one of the preceding claims, wherein the first separation step comprises a mechanical separation step.

6. A process according to claim 5, wherein the mechanical separation step includes a decanting step.

7. A process according to any one of the preceding claims, wherein the first separation step includes a centrifugal separation step.

8. A process according to any preceding claim, wherein the second stream is subjected to a second separation step capable of recovering suspended solids from a liquid, to recover a third stream rich in the fermentation agent and a fourth stream rich in the aqueous solution.

9. A process according to claim 8, wherein the second separation step comprises a mechanical separation step.

10. A process according to claim 9, wherein the mechanical separation step of the second separation stage includes a centrifugal separation step.

11. A process according to claim 10, wherein the centrifugal separation step is carried out using a disk stack separator.

12. A process according to claim 11, wherein the third stream is subjected to a dewatering step.

13. A process according to claim 12, wherein the dewatering step comprises a mechanical dewatering step.

14. A process according to claim 13, wherein the mechanical dewatering step comprises subjecting the third stream to a filter press.

15. A process according to any one of claims 8 to 14, further comprising drying the third stream to reduce the moisture content.

16. A process according to any one of claims 12 to 15, further comprising drying the third stream, after the dewatering step, to further reduce the moisture content.

17. A process according to any one of claims 8 to 16, wherein the fourth stream is subjected to an evaporation step to produce a syrup.

18. A process according to claim 17, wherein the fourth stream is combined with the residual fibre stream.

19. A process according to any one of the preceding claim, wherein the protein rich stream is processed into a cake or pellets.

20. A process according to any preceding claim, wherein the organic material comprises a fermentable carbohydrate.

21. A process according to any preceding claim, wherein the organic material comprises a cereal grain, such as maize, wheat, sorghum or barley; potato; cassava or beet molasses.

22. A process according to any preceding claim, wherein the organic material comprises wheat.

23. A process according to any preceding claim, wherein the fermentation agent comprises fungal cells capable of fermenting the organic material to ethanol.

24. A process according to any preceding claim, wherein the fermentation agent comprises a yeast.

25. A process according to claim 23, wherein the yeast is a yeast of the genus Saccharomyces.

26. A process according to any preceding claim, wherein step (v) is carried out after step (iv) without any intervening treatment step.

27. A process according to any preceding claims, wherein substantially no part of the second stream is combined with the first stream prior to step (v).

28. A process according to any one of claims 1 to 26, when dependent upon claim 8, wherein the fourth stream is combined with the third stream prior to step (v).

29. A process according to any preceding claim, wherein step (v) is carried out after step (iv) without subjecting the first stream to a drying step.

30. A process according to any preceding claim, wherein step (v) is carried out after step (iv) without subjecting the first stream to a heating step.

31. A process according to any preceding claim, wherein step (v) is carried out before any part of the second stream is combined with the first stream.

32. A process according to any preceding claim, further comprising formulating the recovered fermentation agent as a feed additive in an animal feed composition.

33. A process according to any one of the preceding claims, further comprising formulating the contents of the proteinaceous material as a feed additive in an animal feed composition.

34. A process according to any one of the preceding claims, comprising formulating the contents of the proteinaceous material as a protein source in a feed material in an animal feed composition.

35. A process for preparing an animal feed composition, comprising recovering a protein rich stream using a process according to any one of claims 1 to 31 and combining the contents of the protein rich stream with a carbohydrate source, and optionally an additional protein source, and optional additives.

36. An animal feed composition prepared by a process according to claim 35.

37. Apparatus for producing ethanol and a protein-containing fermentation agent comprising:
(i) a fermentation stage for fermenting an aqueous mixture comprising an organic material and a fermentation agent capable of fermenting the organic material, to produce ethanol;
(ii) a first separation stage for recovering the ethanol from the unfermented aqueous mixture;
(iii) a second separation stage, downstream of the first separation stage for recovering unfermented organic material from the fermentation agent and from an aqueous solution of dissolved solids in water; and
(iv) an organic material separation stage for separating at least some protein in the unfermented organic material from the second stage from the rest of the unfermented organic material from the second stage.

38. Apparatus according to claim 37, further comprising:
(v) a third separation stage, downstream of the second separation stage, for recovering the fermentation agent from the aqueous solution.

39. Apparatus according to claim 38, wherein the third separation stage comprises a mechanical separator.

40. Apparatus according to claim 39, wherein the third separation stage comprises a disk stack separator.

41. A protein-containing composition obtainable by a process according to any one of claims 1 to 31.

42. A process for recovering a protein-containing material substantially as herein described with reference to and as shown in the accompanying drawings.

43. Apparatus for producing ethanol, and a protein-containing material substantially as herein described with reference to and as shown in the accompanying drawings.
